(19) United States
(12) Patent Application Publication
(10) Pub. No.: US 2003/0152609 A1
(43) Pub. Date: Aug. 14, 2003

(54) Devices and methods for reducing scar tissue formation

(76) Inventors: Robert E. Fischell, Dayton, MD (US); David R. Fischell, Fair Haven, NJ (US); Tim A. Fischell, Richland, MI (US)

Correspondence Address: Robert E. Fischell, 14600 Viburnum Dr., Dayton, MD, 21036, US

(21) Appl. No.: 10/072,177

(22) Filed: Feb. 11, 2002

(51) Int. Cl. A61L 15/00

(52) U.S. Cl. 424/445

(57) ABSTRACT

Disclosed is a cytostatic drug attached to a sterile sheet that is designed to be placed between internal body tissues to prevent the formation of post-operative adhesions, which adhesions are really scar tissue formation. This sheet onto or into which the drug is placed may be either a permanent implant or it may be biodegradable. By impregnating an existing product such as the Johnson & Johnson SURGICEL™ absorbable hemostat gauze-like sheet with an anti-proliferative drug such as sirolimus, the biodegradable, drug impregnated mesh would act as a barrier to cell proliferation and hence be a deterrent to the formation of adhesions or scar tissue. Another embodiment of this invention is a cytostatic drug attached to a sheet that is placed at the site of an anastamosis to decrease scar tissue formation from within the vessel at the site of the anastomosis.

### FIELD OF USE

This invention is in the field of devices and methods used to prevent the formation of scar tissue that often occurs as a result of a surgical procedure.

### BACKGROUND OF THE INVENTION

Post-operative scar tissue formation, adhesions and blood vessel narrowing are major problems following abdominal, neurological, vascular or other types of surgery. For example, narrowing of a blood vessel at the site of an anastamosis is often caused by the unwanted proliferation of scar tissue at that location.

U.S. patent application Ser. No. 09/772,693 by R. E. Fischell, et al, filed on Jan. 1, 2001 describes various means and methods to reduce scar tissue formation resulting from a surgical procedure. However, this patent application does not describe a cytostatic anti-proliferative surgical wrap that is placed around some human tissue where there is a risk of formation of scar tissue. Although several companies have developed products (such as sheets of biodegradable mesh, gels, foams and barrier membranes of various materials) that can be placed between these structures to reduce the tissue growth, none are entirely effective.

U.S. Pat. No. 5,795,286 describes the use of a beta emitting radioisotope placed onto a sheet of material to reduce scar tissue formation by means of irradiation of the local tissue. Although radioisotopes may be effective at preventing cellular proliferation associated with adhesions, the limited shelf life and safety issues associated with radioisotopes make them less than ideal for this purpose.

Recent publications (Transcatheter Cardiovascular Therapeutics 2001 Abstracts) report a greatly reduced cellular proliferation and reduced restenesis within angioplasty injured arteries when vascular stents used for recannalization are coated with a cytostatic anti-proliferative drug such as Rapamycin (sirolmus), Actinomycin-D or Taxol. However, these drugs have never been used for reducing cellular proliferation at the site of a surgical procedure.

In U.S. Pat. No. 6,063,396, P. J. Kelleher describes the use of highly toxic, antimitotic drugs such as ricin, anthracycline, daunomycin, mitomycin C and dexorubin for reducing scar tissue formation and for wound healing. However, he makes no mention of any cytostatic anti-proliferative drug such as sirolimus or similar acting compounds.

In U.S. Pat. No. 5,981,568 Kunz et al describe the use of certain cytostatic agents that are used to inhibit or reduce restenosis of an artery that is treated from inside that artery. However, Kunz et al does not address the problem of restenosis at an anastamosis which is the surgical connection of two blood vessels. Kunz et al also fails to consider the drug sirolimus or its functional analogs as the drug to be applied for reducing cellular proliferation that can result in scar tissue formation or adhesions.

### SUMMARY OF THE INVENTIONS

One embodiment of this invention is a device consisting of cytostatic anti-proliferative drug impregnated into, coated onto or placed onto a material sheet or mesh designed to be placed generally around human tissue that has been surgically joined or surgically treated; the goal being the prevention of formation of excess post-operative scar tissue. A drug that is impregnated into a suture or gauze-like material or sheet or coated onto the material or joined to the material by adhesion and/or capillary action is defined herein as a drug “attracted” to a suture or mesh or sheet. This suture, mesh or gauze onto which the drug is attached may be either a permanent implant or it may be biodegradable. The drug can be attached to an existing product such as the Johnson & Johnson SURGICEL™ absorbable hemostat gauze-like sheet or a Vicryl mesh product. With a cytostatic anti-proliferative drug such as sirolimus or its functional analogs which have a known effect on proliferating cells, the drug released from the biodegradable mesh would decrease cellular proliferation and hence be a deterrent to the formation of excess scar tissue at the surgical site.

It is also envisioned that a cytostatic anti-proliferative drug could be attached to surgical suture material. This suture/drug combination could be used (for example) to join together two blood vessels; i.e., an anastomosis, with the attached drug causing a reduction in cellular proliferation in the vicinity where the sutures penetrate through the wall of the vessel. A suture material with a cytostatic, antiproliferative drug attached that decreases scar tissue formation would also be useful for sutures in the skin, particularly for plastic surgery. A very important application would be for sutures that are required for eye surgery where reduced scar tissue formation is very much needed. It should be understood that the suture material could be either soluble or insoluble and could be used for any application for which sutures are used.

Still another embodiment of the present invention is a cytostatic anti-proliferative drug coated onto a surgical staple thus reducing scar tissue around that staple.

In addition to applying the cytostatic anti-proliferative drug at the surgical site by means of a device to which the cytostatic anti-proliferative drug is attached, it is also envisioned to apply the least one day from the material onto which they are attachable. In describing this invention, the use of the terms “mesh” or “sheet” or “gel” shall mean the same thing (i.e., a material to which or into which a cytostatic drug is attached) and these words will be used interchangeably. The present invention ideally utilizes those cytostatic drugs, such as sirolimus or Everolimus, that interfere with the initiation of mitosis by means of interaction with TOR protein complex formation and cyclin signaling. These drugs prevent the initiation of DNA replication by acting on cells in close proximity to the mesh from which the drug slowly elutes as very early cell cycle mitosis inhibitors that act at or before the S-phase of cellular mitosis.

Thus it is an object of this invention to have a sheet of material that can be placed into or wrapped generally around some human tissue at the site of a surgical procedure, the material having a cytostatic anti-proliferative drug attached for reducing scar tissue formation at the site of the surgical procedure.

Another object of this invention to have a sheet of material that can be wrapped around a blood vessel, a ureter, a bile duct, a fallopian tube, or any other vessel of the human body at the site of a surgically created anastamosis, the material having a cytostatic anti-proliferative drug attached to reduce scar tissue formation that can result in a narrowing of the vessel or duct at the site of anastamosis.

Still another object of this invention is to have a biodegradable sheet of material or mesh suitable for placement between body tissues including an attached drug that elutes slowly from the sheet of material to prevent cellular proliferation associated with post-surgical adhesions and/or scar tissue formation.

Still another object of the invention is to have a suture material or surgical staple to which a cytostatic anti-proliferative drug is attached.

Still another object of this invention is to have the cytostatic anti-proliferative drug be sirolimus or a functionally equivalent cytostatic and anti-inflammatory drug.

Still another object of the invention is to employ a device placed into the body of a human subject, which device has an attached cytostatic anti-proliferative drug, plus using the same or a different cytostatic anti-proliferative drug as a medication to be applied systemically to the human subject from some time prior to a surgical procedure and/or for some time after that procedure in order to reduce excessive post-surgical scar tissue formation.

These and other objects and advantages of this invention will become obvious to a person of ordinary skill in this art upon reading of the detailed description of this invention including the associated drawings.

### BRIEF DESCRIPTION OF THE DRAWINGS

FIG. 1 illustrates a sheet of material to which a cytostatic anti-proliferative drug has been attached; the sheet is formed so that it can be wrapped around or placed on or between human tissue at the site of a surgical procedure.

FIG. 2 is an enlargement of the cross section of a single strand of the mesh where the drug is embedded within the strand.

FIG. 3 is an enlargement of the cross section of a single strand of the mesh where the drug is coated onto the strand.

FIG. 4 is an enlargement of two strands of the mesh that have been dipped into a solution of a cytostatic anti-proliferative drug thereby attaching the drug to the strands by adhesion and capillary action.

FIG. 5 is a lateral cross section of cytostatic anti-proliferative surgical wrap placed around an end-to-end anastamosis of a vessel or duct.

FIG. 6 is a layout view of the surgical wrap of FIG. 5.

FIG. 7 is a plan view of an annular anti-proliferative sheet for application to anastamoses.

FIG. 8 is a plan view of a annular anti-proliferative sheet for application to anastamoses, the interior of the annulus having slits to facilitate placement onto a connecting blood vessel.

FIG. 9 is a cross section of cytostatic anti-proliferative surgical wrap placed at an aorta-vein graft anastamosis.

FIG. 10 is a cross section of cytostatic anti-proliferative surgical wrap placed at the anastamosis of the internal mammary artery into the side of a coronary artery.

### DETAILED DESCRIPTION OF THE INVENTION

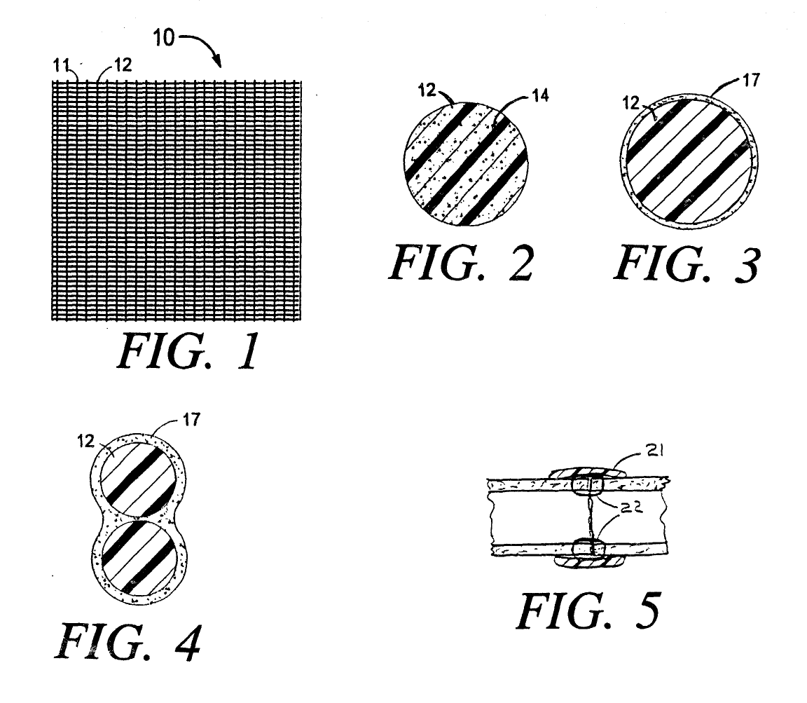

FIG. 1 shows an absorbable mesh sheet 10 with mesh strands 12 and open spaces 11. The sheet 10 is designed to be placed post-operatively into or around human tissue at the site of a surgical procedure. When placed at the site of a surgical procedure, the sheet 10 is designed to slowly elute a cytostatic drug so as to decrease the formation of scar tissue and to reduce the extent of adhesions. When placed generally around human tissue, the mesh 10 forms a cytostatic anti-proliferative surgical wrap. The mesh strands 12 can be made from oxidized regenerated cellulose or other biodegradable materials with the cytostatic anti-proliferative drug either embedded within the strands, coated onto the outer surfaces of the strands or held onto the strands by adhesion or capillary action. Any of these possibilities will be described herein as the drug being attached to the mesh or attached to the strand of the mesh.

FIG. 2 is an enlargement of a cross section of a single strand 12 of the mesh 10 in which the cytostatic anti-proliferative drug 14 is embedded within the strand 12.

FIG. 3 is an enlargement of the cross section of a single strand 12 of the mesh where the cytostatic anti-proliferative drug is placed into a coating 17 formed onto the exterior surface of the strand 12. The strand 12 could be formed from either a biostable or biodegradable polymer material. The material of the coating 17 is selected so that the drug that is placed into the coating 17 will slowly elute into the human tissue at the site of a surgical procedure. To further adjust the rate of release of the drug into adjacent tissue, the coating 17 could be covered with an additional coating (not shown).

FIG. 4 is an enlargement of two adjacent strands 12 of the mesh 10 onto which a cytostatic anti-proliferative drug 18 is attached by means of adhesion and capillary action.

The anti-proliferative drugs that are less suitable for this purpose include cytotoxic cancer drugs such as Taxol, Actinomycin-D, Alkeran, Cytoxan, Leukeran, Cis-platinum, BiCNU, Adriamycin, Doxorubicin, Cerubidine, Idamycin, Mithracin, Mutamycin, Fluorouracil, Methotrexate, Thoguanine, Texotere, Etoposide, Vincristine, Irinotecan, Hycamptin, Matulane, Vumon, Hexalin, Hydroxyurea, Gemzar, Oncovin and Etophophos. The optimum drugs for this purpose do include cytostatic drugs such as sirolimus, anti-sense to c-myc (Resten-NG), tacrolimus (FX506), Everolimus and any other analog of sirolimus including: SDZ-RAD, CCI-770, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy, 2-desmethyl and proline.

Although a mesh has been discussed herein, more generally, a cytostatic anti-proliferative drug can be made to be part of any sheet of material that is or is not biodegradable, as long as the sheet of material is biocompatible. In any case, this material should gradually release the cytostatic anti-proliferative drug into the surrounding surgically injured tissue over a period from as short as a day to as long as a few months. The rate of release being controlled by the type of material into which the drug is placed. It is also envisioned that a polymer coating could be placed over the drug to slow the eluting of the drug into the surrounding tissue. Such polymer materials are well known in the field of slow release of medications, and one example is described in some detail in U.S. Pat. No. 6,143,037 by S. Goldstein et al. The effect of the cytostatic anti-proliferative drug that is attached to at least part of the sheet of material will decrease cellular proliferation and therefore decrease the formation of scar tissue and/or adhesions. Most importantly, such a mesh 10 wrapped around a vascular anastamosis would reduce the narrowing of that vessel which often occurs at the site of the anastamosis.

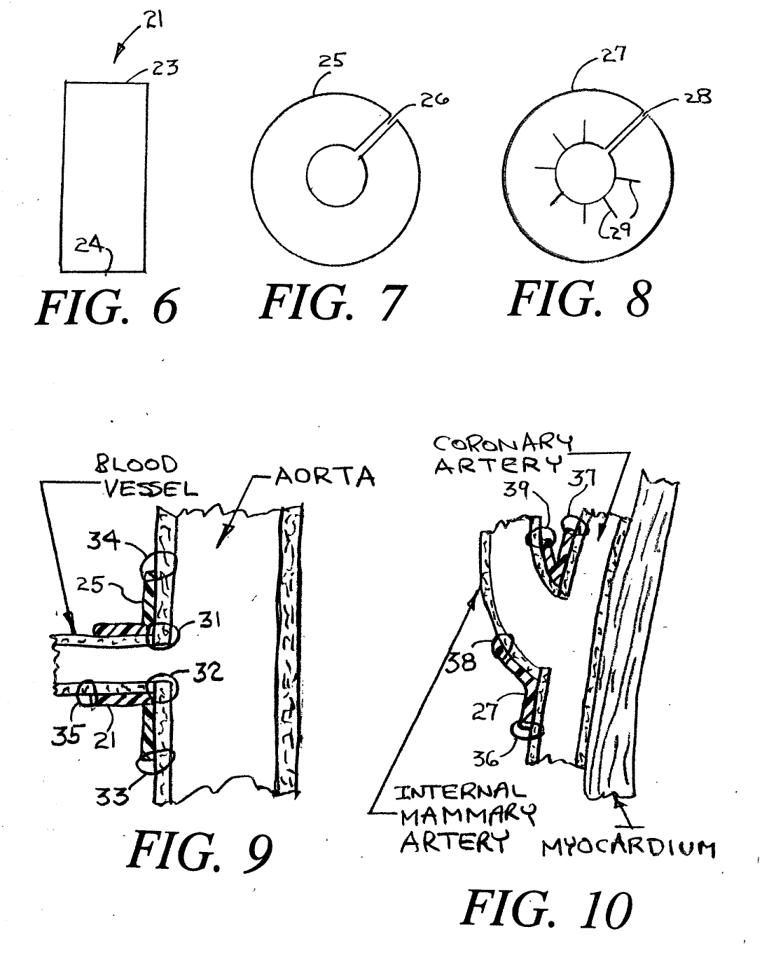

FIG. 5 is a cross section of a cytostatic anti-proliferative surgical wrap 21 shown wrapped around an anastamosis of a vessel or duct, the sutures 22 being used to join the cut ends of the vessel or duct. The vessel or duct can include, but is not limited to, a vein, an artery, the joining of an artificial graft to a vein or artery, a ureter, a urethra, a bile duct, an ileum, a jejunum, a duodenum, a colon or a fallopian tube. Such a wrap could be used anywhere at a site where a surgical procedure has been done. For example, the surgical site might be at the site of operations on the backbone, nerves coming out of a verterbrae, the colon or ileum, etc. A cytostatic anti-proliferative surgical wrap is defined herein as a game-like mesh that is wrapped generally around some human tissue at the site of a surgical procedure. The wrapping could be somewhat more or less than a full 360-degree wrap around the tissue. To accommodate tissues having different diameters, the wrap material could be sterilized in comparatively long lengths and the surgeon could it to the correct length at the time of surgery. This wrap can be sutured in place with either a conventional suture or with sutures to which a cytostatic anti-proliferative drug has been attached. FIG. 6 shows such a wrap 21 having ends 23 and 24, which ends are typically sutured onto the vessel that has an anastamosis.

FIG. 7 shows an annular sheet 25 having a cut 26; the sheet 25 would have an anti-proliferative drug attached to it. The use of this sheet 25 will be explained below with the assistance of FIGS. 9 and 10. FIG. 8 shows a slit annular sheet 27 that has a cut 28 end slits 29. This type of slit annular sheet is particularly well suited for being sutured onto the aorta at the site of an anastamosis with the sections between the slits 29 being placed and sutured onto the blood that is joined to the aorta.

FIG. 9 illustrates a typical anastamosis that occurs during coronary bypass surgery; namely, a blood vessel (typically a vein from the patient's leg) surgically joined to the aorta by sutures 31 and 32. FIG. 9 shows the surgical wrap 21 attached to the blood vessel by means of at least one suture 35. Also shown in FIG. 9 is an annular sheet 25 attached to the aorta by means of sutures 33 and 34. The wrap 21 and sheet 25 would each have attached an anti-proliferative drug as described herein to prevent the formation of scar tissue, within the blood vessel and within the aorta. Such an anastamosis is a frequent site where the formation of scar tissue diminishes the flow of blood through the blood vessel. By the slow release of an anti-proliferative drug attached to the wrap 21 and the sheet 25, there will be a decreased incidence of sterosis at the site of the anastamosis. It should be understood, that either the wrap 21 or the sheet 25, separately or together, could be used at this type of anastamosis.

FIG. 10 illustrates a typical coronary artery bypass graft of an artery or a vein to a coronary artery. FIG. 10 specifically shows an internal mammary artery surgically joined to a coronary artery such as the left anterior descending, left circumflex or right main coronary artery. To avoid the formation of scar tissue inside the anastamosis, a slit annular sheet 27 (as shown in FIG. 8) has been sutured to the coronary artery and the internal mammary artery by means of the sutures 36, 37, 38 and 39. It should be understood that the wrap 21 and/or the sheet 25 could also be applied at this site. Furthermore, the surgeon could cut away some of the sheet located between the slits 29 of the sheet 27 before attaching it by sutures to the site of the anastamosis. Although FIG. 9 shows an anastamosis between the internal mammary artery and a coronary artery, any suitable vein could also be used in place of the internal mammary artery.

Another alternative embodiment of the invention is a suture material to which a cytostatic anti-proliferative drug is attached. A drawing of a highly enlarged cross section of such a suture would be shown by FIGS. 2 or 3. That is, FIG. 2 could be considered to be a cross section of a suture 12 into which is embedded a cytostatic anti-proliferative drug 14. FIG. 3 could be considered a highly enlarged cross section of a suture 12 that is coated with a cytostatic anti-proliferative drug 17. FIG. 5 shows cytostatic anti-proliferative coated sutures 22 used to join a vascular anastamosis. The object of attaching a cytostatic anti-proliferative drug to a suture would be to reduce scar tissue formation where the suture penetrates through human tissue. This would be particularly true for the use a suture to join together two vessels, i.e., an anastamosis. This could be used for both soluble and insoluble suture materials. By using a suture to which a cytostatic anti-proliferative drug is attached, a surgeon would have a method for reducing scar tissue formation on the surface of the skin or anywhere else where sutures are used. A particularly valuable place for such sutures would be for eye or plastic surgery where scar tissue formation can compromise the result of a surgical procedure. Furthermore, a cytostatic anti-proliferative drug could be attached to any surgical staple that is used to join together human tissue after a surgical procedure. It should be understood that sutures or staples with a cytostatic anti-proliferative agent attached could be used for joining any tissue of a human subject where it is desired to reduce cellular proliferation, i.e., the formation of adhesions or scar tissue. It should also be understood that any of the sutures 22, 31, 32, 33, 34, 35, 36, 37, 38 or 39 as shown in FIGS. 5, 9 and 10 could be conventional sutures or could have a cytostatic drug as described herein attached to that suture.

When cytostatic anti-proliferative sutures are used on the skin's surface, it should be understood that an ointment that includes a cytostatic anti-proliferative agent could be applied to the skin at the site of a surgical incision. The cytostatic anti-proliferative agent would be selected from the group that includes sirolimus, anti-sense to c-myc (Resten-NG), tacrolimus (FK506), Everolimus and any other analog of sirolimus including SDZ-RAD, CCI-779, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy, 2-desmethyl and proline.

If an arterio-venus fistula shunt is placed into the arm of a dialyses patient, then the same type of cytostatic anti-proliferative agent(s) as described above could be attached to that shunt device to increase the time during which the associated vein in the arm would remain patent. Ideally, the cytostatic anti-proliferative drug could be placed throughout the inner surface of the shunt or it could be placed near the ends where the shunt attaches to the vein or to the artery.

For any of the applications described herein, the systemic application of one or more of the cytostatic anti-proliferative agents that have been described could be used conjunctively to further minimize the creation of scar tissue.

Although only the use of certain cytostatic anti-proliferative agents has been discussed herein, it should be understood that other medications could be added to the cytostatic anti-proliferative drugs to provide an improved outcome for the patients. Specifically, for applications on the skin, an antiseptic, and/or anti-biotic, and/or analgesic, and/or anti-inflammatory agent could be added to a cytostatic anti-proliferative ointment to prevent infection and/or to decrease pain. These other agents could also be applied for any other use of the cytostatic anti-proliferative drugs that are described herein it is further understood that any human subject in whom a cytostatic anti-proliferative agent is used plus at least one of the other drugs listed above could also benefit from the systemic administration of one or more cytostatic anti-proliferative agent that has been listed herein.

Various other modifications, adaptations, and alternative designs are of course possible in light of the above teachings. Therefore, it should be understood at this time that within the scope of the appended claims, the invention can be practiced otherwise than as specifically described herein.

What is claimed is:

1. A cytostatic anti-proliferative surgical wrap sheet of material adapted for being wrapped generally around tissues of a human body at the site of a surgical procedure, the sheet of material having a cytostatic anti-proliferative drug attached, the action of the cytostatic anti-proliferative drug being a reduction in the generation of scar tissue, the cytostatic anti-proliferative drug being selected from the group that includes sirolimus, anti-sense to c-myc (Resten-NG), tacrolimus (FK506), Everolimus and the any functional analog of sirolimus including: SDZ-RAD, CCI-779, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy, 2-desmethyl and proline.

2. The cytostatic anti-proliferative surgical wrap of claim 1 wherein the sheet of material is drug eluting and biodegradable.

3. The cytostatic anti-proliferative surgical wrap of claim 1 wherein the sheet of material is in the form of a mesh that is drug eluting and biostable.

4. The cytostatic anti-proliferative surgical wrap of claim 1 further including at least one additional medication attached to the wrap, the medication being selected from the group that includes an anti-biotic medication, an anti-inflammatory medication or an analgesic medication.

5. In combination, a cytostatic anti-proliferative drug attached to a surgical suture, the suture being adapted to connect human tissue that is separated by a surgical procedure on a human subject, the cytostatic anti-proliferative drug being selected from the group that includes sirolimus, anti-sense to c-myc (Resten-NG), tacrolimus (FK506), Everolimus and the following functional analogs of sirolimus: SDZ-RAD, CCI-779, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy, 2-desmethyl and proline.

6. In combination, a cytostatic anti-proliferative drug attached to a sheet for placement at an anastamosis of a vessel of the human body, the drug and sheet combination being adapted to prevent narrowing of the vessel at the site of the anastamosis of that vessel, the cytostatic anti-proliferative drug being selected from the group that includes sirolimus, anti-sense to c-myc (Resten-NG), tacrolimus (FK506), Everolimus or any other functional analog of sirolimus including: SDZ-RAD, CCI-779, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy, 2-desmethyl and proline.

7. The combination of claim 6 wherein the sheet is in the form of a flat rectangle that is adapted to be placed around a vessel at the site of the anastamosis.

8. The combination of claim 6 wherein the sheet is in the form of an annulus.

9. A means for improving the outcome of a surgical procedure on a human subject, the means being the systemic release into the human subject on whom the surgical procedure has been performed of a cytostatic anti-proliferative agent in combination with at least one other drug selected from the group that includes antiseptic agents, anti-biotic agents and analgesic agents, the cytostatic anti-proliferative agent being selected from the group that includes sirolimus, anti-sense to c-myc (Resten-NG), tacrolimus (FK506), Everolimus and other analog of sirolimus including: SDZ-RAD, CCI-779, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy, 2-desmethyl and proline.

10. The means of claim 9 wherein additionally the cytostatic anti-proliferative agent is used in an ointment that is applied to the skin.

11. The means of claim 9 wherein additionally the cytostatic anti-proliferative agent is attached to a mesh that is adapted to be placed within the human subject in whom the surgical procedure was performed.

12. The means of claim 9 wherein the cytostatic anti-proliferative agent is attached to a suture.

13. A method for decreasing the formation of scar tissue after a surgical procedure, the method comprising the following steps:
a) attaching a cytostatic anti-proliferative drug onto a mesh that is adapted for placement generally around tissue of a human subject; the antiproliferative drug being selected from the group that includes sirolimus, anti-sense to c-myc (Resten-NG), tacrolimus (FK506), Everolimus and any other analog of sirolimus including: SDZ-RAD, CCI-779, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy, 2-desmethyl and proline.
b) placing the mesh with attached cytostatic anti-proliferative drug generally around tissue of the human subject during or after completing a surgical procedure.

14. The method of claim 13 wherein the surgical procedure includes the forming of an anastamosis of a vessel of the human subject, the vessel being selected from the group that includes an artery, a vein, a ureter, a urethra, an artificial graft, a jejunum, an ileum, a duodenum, a colon, a bile duct or a fallopian tube.

15. The method of claim 13 further including the step of systemic application into the human subject of at least one cytostatic anti-proliferative drug at least one day prior to the surgical procedure.

16. The method of claim 13 further including the step of a continuing systemic application into the human subject of at least one cytostatic anti-proliferative drug for at least one day after the surgical procedure.

17. The method of claim 13 wherein the surgical procedure is the creation of an anastamosis to join a vein to the aorta of the human subject.

18. The method of claim 13 wherein the surgical procedure is the creation of an anastamosis to join an internal mammary artery to a coronary artery of the human subject.

19. A method for decreasing scar tissue formation on a cut in the skin of a human subject, the method comprising the following steps:
a) placing an ointment onto the skin at the site of the cut, the ointment including a cytostatic anti-proliferative agent selected from the group that includes sirolimus, anti-sense to c-myc (Resten-NG), tacrolimus (FK506), Everolimus or any other functional analog of sirolimus including: SDZ-RAD, CCI-779, 7-epi-rapamycin, 7-thiomethyl-rapamycin, 7-epi-trimethoxyphenyl-rapamycin, 7-epi-thiomethyl-, 7-demethoxy-rapamycin, 32-demethoxy, 2-desmethyl and proline; and
b) placing a bandage over the ointment, the bandage being attached to the skin in the vicinity of the cut.

20. The method of claim 19 including the step of placing into the ointment at least one additional therapeutic agent that is selected from the group that includes an antiseptic drug, an anti-biotic drug and an analgesic drug.

21. The method of claim 19 further including the step of the systemic administration of at least one cytostatic anti-proliferative agent selected from the group that includes sirolimus, anti-sense to c-myc (Resten-NG), tacrolimus (FK506), Everolimus and any other functional analog of sirolimus including: SDZ-RAD, CCI-779, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy, 2-desmethyl and proline.

22. A sheet for placement at or near the site of a surgical procedure to reduce the formation of scar tissue and adhesions, the sheet including a cytostatic drug that is released from the mesh or sheet over a period that is longer than a day, the cytostatic drug being capable of preventing the initiation of DNA replication of cells in the vicinity of the mesh by acting as a cell cycle mitosis inhibitor that acts on the cells in the vicinity of the mesh at or before the S-phase of cellular mitosis.
